(19) United States
(12) Patent Application Publication
(10) Pub. No.: US 2021/0338719 A1
(43) Pub. Date: Nov. 4, 2021

(54) COMPOSITION COMPRISING FERROUS SULPHATE MONOHYDRATE AND LONG CHAIN POLYUNSATURATED FATTY ACIDS

(71) Applicant: SOCIETE DES PRODUITS NESTLE S.A., Vevey (CH)

(72) Inventors: Joeska Husny, Bern (CH); Maithieu Bedard, Thun (CH)

(21) Appl. No.: 16/958,436

(22) PCT Filed: Dec. 21, 2018
(86) PCT No.: PCT/EP2018/086687

§ 371 Date: Jun. 26, 2020

(30) Foreign Application Priority Data
Dec. 28, 2017 (EP) 17210915.9

(51) Int. Cl. A61K 33/26; A61K 31/232

(52) U.S. Cl. CPC A61K 33/26; A61K 31/232

(57) ABSTRACT

The present invention relates to a composition comprising long chain polyunsaturated fatty acids (LC-PUFA) and ferrous sulphate monohydrate. Ferrous sulphate monohydrate advantageously does not cause significant oxidation of LC-PUFAs.

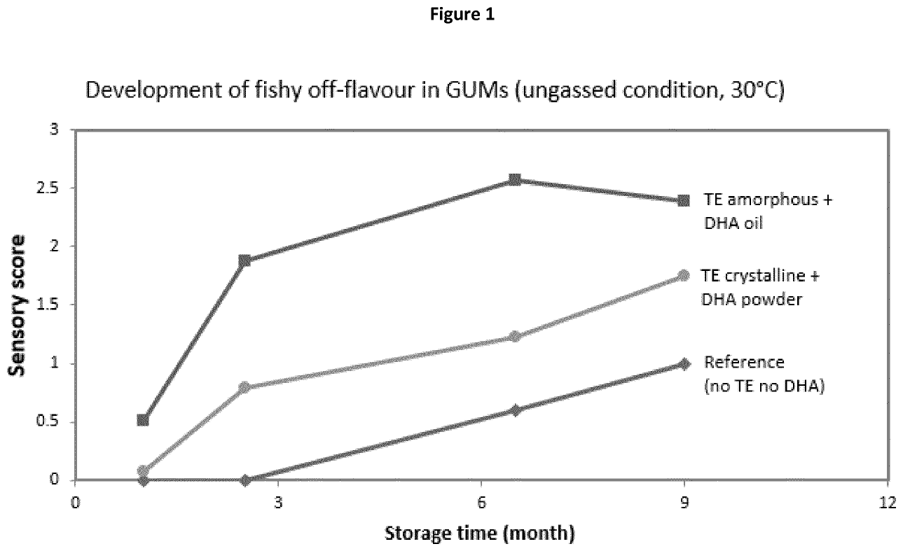

### CROSS REFERENCE TO RELATED APPLICATIONS

The present application is a National Stage of International Application No. PCT/EP2018/086687, filed on Dec. 21, 2018, which claims priority to European Patent Application No. 17210915.9, filed on Dec. 28, 2017, the entire contents of which are being incorporated herein by reference.

### FIELD OF THE INVENTION

The present invention relates to a composition comprising long chain polyunsaturated fatty acids (LC-PUFA) and ferrous sulphate monohydrate. Ferrous sulphate monohydrate advantageously does not cause significant oxidation of LC-PUFAs.

### BACKGROUND OF THE INVENTION

Food products and beverages, comprise a wide variety of nutrients, which may have negative interactions with each other. This is typically the case when iron is present in a composition together with compounds sensitive to oxidation, such as LC-PUFA, as iron tends to oxidize such compounds, leading to undesired modification of the sensory and/or nutritional properties of such compounds.

Iron is a particularly important micro-nutrient. Worldwide, iron deficiency is one of the most prevalent nutrient deficiencies. In humans, iron is essential for the functioning of a large number of biological processes such as oxygen binding and transport, gene regulation, neurological function, immune function and regulation of cell growth and differentiation. Iron deficiency may result in anaemia, as well as a variety of health problems, such as impairment of thyroid, immune and mental functions, physical performance, cognitive development, increased sensitivity to insulin and fatigue. Iron deficiency is especially widespread in pregnant and lactating women, as well as in infants and children.

Fortification of foods with iron is one approach to combatting iron deficiency. Therefore, the inclusion of an added iron source in dietary compositions or supplements, particularly dietary compositions or supplements for infants, small children, women pre-pregnancy, during pregnancy and/or during lactation, is highly desirable. Diverse iron compounds have been used as iron fortifying agents in food products and in nutritional supplements. For example, ferrous sulphate is widely used, owing to its relatively low price and high bioavailability.

However, the present inventors have found that a number of iron compounds, when used to fortify a composition, have a deleterious effect on compounds sensitive to oxidation such as LC-PUFAs.

LC-PUFAs are essential components of our diet and scientific evidence supports that specific LC-PUFAs are important for brain and retina development, heart health and a number of other health benefits. Vitamins are also essential nutrients, which are necessary for the prevention of numerous diseases and disorders. Polyphenols are also associated with health benefits and are for example associated with prevention of degenerative diseases, cardiovascular disease and cancer.

However, compounds such as LC-PUFAs oxidize in the presence of oxygen, especially in the presence of iron. Lipid oxidation influences the quality of food products through flavour and taste deterioration and reduction in nutritional value. Off-flavour and off-taste formation such as rancidity, fishiness, metallic, fried fat, etc, results mainly from the degradation of primary oxidation products of LC-PUFA, such as peroxides, which can readily isomerise and degrade to produce volatile compounds. The deterioration of sensory properties is a major cause of consumer complaints in the food industry. Furthermore, shelf-life can be significantly impaired upon oxidation of sensitive compounds.

As a result of a growing interest for enrichment of food with LC-PUFAs bringing significant nutritional benefits, a lot of work has been reported on the development of technologies able to reduce degradation of such compounds.

Focus has been put on masking agent and flavour for avoiding the fishy off notes generated by lipid oxidation in food matrices. However, flavour & masking agents do not stabilize the lipids such as LC-PUFAs, consequently the resulting oxidation leads to a reduction of the nutritional value.

Appropriate process & packaging should also decrease the rate of oxidation of sensitive compounds in food matrices e.g. via a separation of sensitive compounds or iron from the rest of the food matrix. However, this solution is really expensive and not applicable for every type of product.

Some solutions are based on ingredients able to stabilize sensitive compounds, such as encapsulation technologies or specific antioxidants. However, these solutions are preferably specific for the selected type of food matrices, and are tailored around one specific encapsulated ingredient only.

Specific iron sources have been found having reduced oxidative impact on LC-PU FAs. This is for example the case of ferric saccharate (WO 2015/097113). Ferric saccharate is however less bioavailable than for example ferrous sulphate. WO00/51446 has also described complexes formed of ferric ions and caseinate, which had good stability, caused little oxidation of sensitive compounds and had good bioavailability. However such complexes have the significant drawback of forming precipitates at high levels of iron addition and of forming haze when used in transparent beverages and solutions. The different drawbacks of the solutions provided by the prior art show that it is difficult to find iron sources having reduced oxidative potential, while having good bioavailability and while being soluble and providing good sensory attributes to the product in which they are incorporated.

Thus, an object of the present invention is to provide compositions comprising high amounts of an added iron source and high amounts of LC-PUFAs, in which oxidation of the LC-PUFAs by iron is minimized, while providing a highly bioavailable iron source.

### SUMMARY OF THE INVENTION

The present inventors have surprisingly found that ferrous sulphate, when used as iron source in a composition containing LC-PUFAs, does not cause significant oxidation of the LC-PUFAs.

In a first aspect, the invention provides a composition comprising LC-PUFAs and an iron source, characterized in that the iron source is ferrous sulphate monohydrate.

In a second aspect, the invention relates to the use of an iron source for the fortification of a composition comprising LC-PUFAs, characterized in that the iron source is ferrous sulphate monohydrate.

In a third aspect, the invention provides a method for providing a nutrition to an individual comprising feeding the individual with an edible composition of the invention.

In a fourth aspect, the invention provides an edible composition comprising LC PUFAs and an iron source, for use in the prevention, reduction and/or treatment of iron deficiency in an individual, characterized in that the iron source is ferrous sulphate monohydrate.

In an fifth aspect, the invention provides a method for reducing and/or preventing the oxidation of LC-PUFAs in a composition comprising an added iron source, characterized in that ferrous sulphate monohydrate is used as the added iron source.

In a sixth aspect, the invention provides a composition of the invention for use in the prevention, amelioration or treatment of malnutrition, metabolic diseases and/or neuro-degenerative diseases.

In a seventh aspect, the invention provides a composition of the invention for use in the promotion of the development of the nervous system and/or of the retina, in the promotion and/or improvement of the mental performance, behavioural and visual functions of an infant or a child, for strengthening immunity, including the development of gut microflora, and/or for reducing the risk of the development of overweight, obesity and insulin resistance.

### DETAILED DESCRIPTION OF THE INVENTION

### Definitions

The term “iron” is herein intended as designating the ion Fe²⁺, unless otherwise specified.

An “added iron source” is intended for the purpose of the present invention as a ferrous or ferric compound added to the composition for the sole benefit of iron supplementation. Depending on its nature, the composition may comprise iron coming from other ingredients, for example from milk, fruit, vegetable, cereal or fibre components. Iron present in such ingredients is not intended here as an “added iron source”, because it is inherently present in an ingredient that is not primarily added for its iron content, but for its overall nutritional value.

An iron source is intended for the purpose of the present invention as being “substantially the only added iron source” in the composition, provided that other added iron sources are used in a sufficiently small amount not to cause statistically significant oxidation of LC-PUFAs. The skilled person can assess whether a statistically significant loss of LC-PUFAs is caused by applying the method described in the examples of the present application and applying commonly known statistical techniques for the analysis of the results.

The term “nutritional composition” designates a product intended to provide a complete nutrition or a supplemental nutrition to an individual (i.e. to fulfil essential nutritional needs of such individual) and in which the prominent objective is to provide nutrition. A nutritional composition aims at providing specific nutrients to an individual having special nutritional needs, such as infants, young children, pregnant or lactating women, elderly people or people with adverse medical condition requiring special food (e.g. tube feeding compositions or compositions for paediatric subjects). Products in which the hedonic aspect is prominent and nutritional qualities are not of primary importance are excluded from the “nutritional products”. Nutritional compositions preferably comprise proteins, fats, carbohydrates and diverse micro-nutrients.

In the present invention, the term “infant” means a child between birth and 12 months of age. The terms “young child” refer to a child between 12 months and 5 years of age, preferably between 12 months and 3 years of age.

The expression “infant formula” as used herein refers to a foodstuff intended for particular nutritional use by infants and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The infant formulas can encompass the starter infant formulas and the follow-up or follow-on formulas. Generally a starter formula is for infants from birth as breast-milk substitute. A follow-up or follow-on formula is given from the 6th month onwards. It constitutes the principal liquid element in the progressively diversified diet of this category of person. It is to be understood that infants can be fed solely with infant formulas, or that the infant formula can be used as a supplement or complement of human milk.

The “growing-up milks” (or GUMs) are given from one year onwards. It is generally a milk-based beverage adapted for the specific nutritional needs of young children.

The expression “baby food” means a foodstuff intended for particular nutritional use by infants or children such as young children, during the first years of life.

The expression “infant cereal composition” means a cereal-based foodstuff intended for particular nutritional use by infants or children such as young children, during the first years of life.

The term “fortifier” refers to nutritional compositions suitable for mixing with breast milk or infant formula. The “breast milk” should be understood as the mother's milk or the colostrum of the mother or a donor's milk or the colostrum of a donor's milk.

The term “supplement” refers to a composition that can be used to supplement, or complement, the nutrition of an individual.

The term “prebiotic” means non-digestible carbohydrates that beneficially affect the host by selectively stimulating the growth and/or the activity of healthy bacteria in the colon of humans (Gibson G R, Roberfroid M B. Dietary modulation of the human colonic microbiota: introducing the concept of prebiotics. J Nutr. 1995; 125:1401-12).

As used herein, the term “probiotic bacteria” refers to bacterial cell preparations with a beneficial effect on the health or well-being of the host [Salminen S, et al., “Probiotics: how they should be defined”, Trends Food Sc. Technol, (1999), 10, 107-10].

### Composition

The composition of the present invention comprises LC-PUFAs and an iron source, said iron source being ferrous sulphate monohydrate.

Ferrous sulphate exists in various forms of hydration (mono-, tetra-, penta-, hexa- and heptahydrate). The tetra-, penta- and hexahydrate forms being unstable, they are rarely used commercially. Ferrous sulphate monohydrate (also sometimes designated as “dried”) and ferrous sulphate heptahydrate are both stable and crystalline forms of ferrous sulphate that are commonly used commercially.

Ferrous sulphate is also commonly used in powdered products in the form of a spray-dried powder formed by spray-drying dissolved ferrous sulphate in a carrier, such as maltodextrin (hereinafter referred to as “dissolved ferrous sulphate in spray-dried form”). In this case, the ferrous sulphate is typically dissolved at acidic pH, such as pH2, before being admixed with a carrier and dried. In dissolved ferrous sulphate in spray-dried form, the iron and sulphate ions remain dissociated from each other and are dispersed in the amorphous carrier.

Now, the present inventors have found that ferrous sulphate heptahydrate and dissolved ferrous sulphate in spray-dried form both cause significant oxidation of LC-PUFAs, whereas no significant oxidation of LC-PUFAs is observed with the monohydrate form of ferrous sulphate. Without wishing to be bound by theory, the present inventors believe that its crystalline state and its low level of hydration both significantly delay the dissolution of ferrous sulphate monohydrate in aqueous media.

This positive effect is observed when ferrous sulphate monohydrate is used as such and when ferrous sulphate monohydrate is dispersed in an amorphous matrix, provided that the crystalline structure and the level of hydration of ferrous sulphate monohydrate remain. Such ingredient with ferrous sulphate monohydrate dispersed in a matrix can be obtained by mixing the crystalline iron salt in a carrier solution and spray-drying the carrier. The process shall be carried out without dissolving the crystalline iron salt by keeping a sufficiently high pH, preferably by keeping a non-acidic pH. The person skilled in the art can routinely assess if a product or ingredient comprises ferrous sulphate monohydrate with its crystalline structure and its level of hydration. Indeed, several analytical techniques can be employed for identification of ferrous sulphate monohydrate, including polarised microscopy and NIR spectroscopy.

The use of ferrous sulphate monohydrate is particularly advantageous, in that it is characterized at the same time by good bioavailability and by causing only limited oxidation of LC-PUFAs. Ferrous sulphate monohydrate is commercially available, for example from Dr. Paul Lohmann GmbH KG, Emmerthal, Germany or DSM Nutritional Products, Heerlen, the Netherlands.

Now, the present inventors have found that most iron sources, such as the widely used ferrous sulphate heptahydrate and dissolved ferrous sulphate in spray-dried form, cause significant oxidation of sensitive compounds such as LC-PUFAs, whereas significantly reduced oxidation is caused by ferrous sulphate monohydrate.

The use of ferrous sulphate monohydrate is particularly advantageous, in that it is characterized at the same time by good bioavailability and by low oxidative potential. It has been shown that ferrous sulphate monohydrate is characterized by the same bioavailability as ferrous sulphate heptahydrate, which is the golden standard in terms of bioavailability in human.

In a preferred embodiment, at least 50 wt %, more preferably at least 60 wt %, more preferably at least 70 wt %, more preferably at least 80 wt %, even more preferably at least 90 wt % of the added iron is in the form of a ferrous sulphate monohydrate. Even more preferably, ferrous sulphate monohydrate is substantially the only added iron source in the composition. Most preferably, ferrous sulphate monohydrate is the only added iron source in the composition.

The added iron source is preferably present in an amount such as to provide from 6 to 50 mg, preferably 6 to 20 mg, more preferably 6 to 18 mg, more preferably 6 to 15 mg, or from 8 to 20 mg, preferably 8 to 18 mg, more preferably 8 to 15 mg of iron, per 100 g of composition, based on the total dry weight of the composition.

Preferred LC-PUFAs comprise docosahexaenoic acid (DHA, fatty acid 22:6n-3) and eicosapentaenoic (EPA, fatty acid 22:5n-3). The most preferred LC-PUFA is DHA. Suitable sources of LC-PUFA include fish oil and microbial oil, such as microalgae oil. The LC-PUFAs are preferably present in the composition in an amount of 10 to 1000 mg, such as 10 to 750 mg, preferably 50 to 600 mg, more preferably 100 to 600 mg, even more preferably 200 to 600 mg, or in an amount of 10 to 500 mg, preferably 50 to 500 mg, more preferably 100 to 500 mg, most preferably 200 to 500 mg, of LC-PUFA per 100 g of composition based on the total dry weight of the composition.

In a preferred embodiment, the iron is present in the composition in an amount of 0.1 to 10 g, preferably 0.5 to 10 g, more preferably 1 to 10 g, even more preferably 2 to 10 g, most preferably 0.1 to 8 g, or 0.1 to 6 g, preferably 0.1 to 5 g, or 1 to 8 g, preferably 1 to 6 g, more preferably 1 to 5 g, most preferably 1 to 2.5 g of iron per 100 g of LC-PUFA, preferably per 100 g of DHA. Such high amounts of iron per 100 g of LC-PUFA, preferably per 100 g of DHA, are a real challenge and are rendered possible by the particularly low oxidation potential of ferrous sulphate monohydrate.

In preferred embodiment, the LC-PUFAs are encapsulated. Preferably it is microencapsulated. Such LC-PUFAs are preferably in whole or in part encapsulated in a glassy matrix of dairy proteins and glucose. Such a glassy matrix of dairy proteins and glucose can be prepared from any dairy protein available and suitable for this purpose, e.g. whey protein, casein, caseinate, milk proteins, β-lactoglobulin, α-lactalbumin, etc. Encapsulation may be carried out using techniques known in the art. Preferably LC-PUFA is encapsulated in a glassy matrix of dairy proteins and glucose as described in WO 2011/008097 A1 of Friesland Brands B.V., NL or can be obtained from FrieslandCampina Kievit under the trade name NIF powder.

The present inventors have particularly found that the combination of ferrous sulphate monohydrate as a source of iron and LC-PUFA encapsulated in a glassy matrix of dairy proteins and glucose was particularly effective in reducing the oxidation of LC-PUFAs. Such combination is thus particularly advantageous for the purpose of the present invention.

The composition may be in liquid or in powder from. Preferably it is in powder form. When the composition is in powder form, it may be in the form of free powder or in the form of compressed powder, such as in the form of a tablet. Preferably the composition in powder form is not intended to be used in the form of a powder, but is to be reconstituted in a liquid, preferably in an aqueous liquid, most preferably in water, before use.

Preferred compositions of the invention include a food or beverage product, an animal feed product, a nutritional supplement for human or animal, a pharmaceutical composition or a cosmetic composition.

In another preferred embodiment, the composition is an edible composition. Food and beverage products include all products intended to be consumed orally by human beings, for the purpose of providing nutrition and/or pleasure. In a preferred embodiment, the product is a nutritional composition. More preferably it is a nutritional composition selected from an infant formula, a growing-up milk, a baby food, an infant cereal composition, a fortifier, a supplement and a nutritional composition for pregnant or lactating women. More preferably, it is selected from an infant formula, a growing-up milk, an infant cereal composition and a nutritional composition for pregnant or lactating women. Nutritional compositions for pregnant or lactating women are particularly preferred, as these products often comprise particularly high amounts of LC-PUFAs and iron.

The product can also be in the form of an animal feed product or a nutritional supplement for animals. Preferably, the animal is a mammal. Examples of animals include primates, cows, sheep, goats, horses, dogs, cats, rabbits, rats, mice, fish, birds and the like.

Nutritional supplements are intended to be consumed as such or to be added to food or to a beverage. Such supplements are intended to provide additional nutrients and/or a health benefit to the subject consuming it, as well as other beneficial ingredients, including LC-PUFA, and iron. A supplement according to the present invention can be used for providing nutrients and/or a health benefit to human beings, as well as to animals, as defined above. Nutritional supplements include for example supplements to be added to breast milk, for example for premature or low birth weight infants. It also includes supplements for women pre-pregnancy, during pregnancy and/or during lactation.

Pharmaceutical compositions are compositions intended to treat or to prevent an adverse medical condition in a subject in need thereof.

Cosmetic compositions are typically intended for an aesthetic effect on the body and may preferably be administered by oral route.

The composition, preferably the nutritional composition, preferably comprises protein, carbohydrates, fats, vitamins and/or other minerals. Preferably, it comprises all of these types of nutrients.

The proteins may be intact or hydrolysed (extensively or partially hydrolysed).

The nutritional composition according to the present invention generally contains a source of lipids, in addition to the LC-PUFAs. This is particularly relevant if the nutritional composition of the invention is an infant formula. In this case, the lipid source may be any lipid or fat which is suitable for use in infant formulae, in products for small children and/or in products for women during pregnancy, during lactation and pre-pregnancy. Some suitable fat sources include palm oil, high oleic sunflower oil and high oleic safflower oil. The essential fatty acids linoleic and α-linolenic acid may also be added.

The composition according to the present invention may contain a carbohydrate source, such as lactose, maltodextrin, starch and mixtures thereof. The composition according to the present invention may also contain a particular type of carbohydrates: prebiotics. The prebiotics that may be used in accordance with the present invention are not particularly limited and include all food substances that promote the growth of probiotics or health beneficial micro-organisms in the intestines. Preferably, they may be selected from the group consisting of oligosaccharides, optionally containing fructose, galactose, and mannose; dietary fibers, in particular soluble fibers, soy fibers; inulin; or mixtures thereof. Some examples of prebiotics are fructo-oligosaccharides (FOS), galacto-oligosaccharides (GOS), isomalto-oligosaccharides (IMO), xylo-oligosaccharides (XOS), arabino-xylo oligosaccharides (AXOS), mannan-oligosaccharides (MOS), inulin, polydextrose, glycosylsucrose (GS), lactosucrose (LS), lactulose (LA), palatinose-oligosaccharides (PAO), malto-oligosaccharides, gums and/or hydrolysates thereof, pectins and/or hydrolysates thereof. In a particular embodiment, the prebiotics may be fructooligosaccharides and/or inulin. Suitable commercial products that can be used include combinations of FOS with inulin such as the product sold by BENEO under the trademark Orafti, or polydextrose sold by Tate & Lyle under the trademark STA-LITE®.

The prebiotics can also be a BMO (bovine's milk oligosaccharide) and/or a HMO (human milk oligosaccharide) such as N-acetylated oligosaccharides, sialylated oligosaccharides, fucosylated oligosaccharides and any mixtures thereof.

A particular example of prebiotic is a mixture of galacto-oligosaccharide(s), N-acetylated oligosaccharide(s) and sialylated oligosaccharide(s) in which the N-acetylated oligosaccharide(s) represent 0.5 to 4.0 wt % of the oligosaccharide mixture, the galacto oligosaccharide(s) represent 92.0 to 98.5 wt % of the oligosaccharide mixture and the sialylated oligosaccharide(s) represent 1.0 to 4.0 wt % of the oligosaccharide mixture. For example a composition for use according to the invention can contain from 2.5 to 15.0 wt % CMOS-GOS on a dry matter basis with the proviso that the composition comprises at least 0.02 wt % of an N-acetylated oligosaccharide, at least 2.0 wt % of a galacto-oligosaccharide and at least 0.04 wt % of a sialylated oligosaccharide. WO2006087391 and WO2012160080 provide some examples of production of such an oligosaccharide mixture.

The composition may also comprise probiotics microorganisms, preferably probiotic bacteria. Any probiotic bacteria can be used in the composition of the invention, preferably live probiotic bacteria. The composition of the invention advantageously comprises live probiotic bacteria in addition to the at least one compound sensitive to oxidation, because the iron-casein complex described herein has been shown not to be detrimental to the viability of probiotic bacteria, contrary to many iron sources, such as the commonly used ferrous sulphate heptahydrate and dissolved ferrous sulphate in spray-dried form. In embodiments where probiotic bacteria are present together with the compound sensitive to oxidation, the composition of the present invention is preferably in powder form and more preferably it is a composition in powder form to be reconstituted with a liquid such as water.

Examples of probiotic bacteria that can be present in the composition of the present invention include bifidobacteria, lactobacilli, lactococci, enterococci, streptococci, Leuconostoc, Escherichia, propionibacteria, or combinations thereof, preferably it is a bacteria of the Lactobacillus or of the Bifidobacterium genus.

Preferably the probiotic bacteria is selected among the species Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus rhamnosus, Lactobacillus johnsonii, Lactobacillus plantarum, Lactobacillus fermentum, Lactobacillus reuteri, Lactococcus lactis, Streptococcus thermophilus, Lactococcus diacetylactis, Lactococcus cremoris, Lactobacillus delbrueckii subsp. bulgaricus, Lactobacillus delbrueckii subsp. lactis, Lactobacitlus helveticus, Escherichia coli, Enterococcus faecium, Leuconostoc pseudomesenteroides, Bifidobacterium bifidum, Lactobacillus gasseri, Lactobacillus sakei, Streptococcus salivarius, as well as any of their subspecies and/or mixtures thereof.

More preferably, it is selected from the species Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacittus salivarius, Lactobacillus rhamnosus, Lactobacillus johnsonii, Lactobacillus plantarum, Lactobacillus fermentum, Lactobacillus routeri, Lactobacillus delbrueckii subsp. bulgaricus, Lactobacillus delbrueckii subsp. lactis, Lactobacillus helveticus, Bifidobacterium bifidum, Lactobacillus gasseri, Lactobacillus sakei and mixtures thereof.

Examples of bacterial strains that can advantageously be present in the composition include Bifidobacterium longum (deposited as ATCC BAA-999), Bifidobacterium longum (deposited as CNCM 1-2618), Bifidobacterium breve (deposited as CNCM 1-3865), Bifidobacterium lactis (deposited as CNCM 1-3446), Lactobacillus johnsonii (deposited as CNCM 1-1225), Lactobacillus paracasei (deposited as CNCM 1-2116), Lactobacillus rhamnosus (deposited as CGMCC 1.3724), Streptococcus thermophilus (deposited as CNCM 1-1422), Streptococcus thermophilus (deposited as CNCM 1-4153), Streptococcus thermophilus (deposited as CNCM 1-1985), Streptococcus thermophilus (deposited as CNCM 1-3915), Lactobacillus casei (deposited as CNCM 1-1518), Lactobacillus casei (deposited as ACA-DC 6002), Escherichia coli Nissle (deposited as DSM 6601), Lactobacillus bulgaricus (deposited as CNCM 1-1198), Lactococcus lactis (deposited as CNCM 1-4154), or combinations thereof.

More preferred bacterial strains include Bifidobacterium longum (deposited as ATCC BAA-999), Bifidobacterium longum (deposited as CNCM 1-2618), Bifidobacterium breve (deposited as CNCM 1-3865), Bifidobacterium lactis (deposited as CNCM 1-3446), Lactobacillus johnsonii (deposited as CNCM 1-1225), Lactobacillus paracasei (deposited as CNCM 1-2116), Lactobacillus rhamnosus (deposited as CGMCC 1.3724), Lactobacillus casei (deposited as CNCM I-1518), Lactobacillus casei (deposited as ACA-DC 6002), Streptococcus thermophilus (deposited as CNCM 1-3915) and Lactobacillus bulgaricus deposited as (CNCM I-1198) or combinations thereof.

In a further preferred embodiment the probiotic bacteria is selected from Bifidobacterium longum (deposited as ATCC BAA-999), Lactobacillus rhamnosus (deposited as CGMCC 1.3724) and Lactobacillus paracasei (deposited as CNCM 1-2116) and mixtures thereof.

The probiotic bacteria is preferably present in the composition in an amount of at least 5E+06 CFU per gram of composition, on a dry weight basis, preferably 5E+06 to 1 E+12 CFU per gram of composition, more preferably 5E+06 to 5E+11 CFU per gram of composition, most preferably 5E+06 to 5E+10 CFU per gram of composition.

The selected probiotic bacteria may be cultured according to any suitable method and prepared for addition to the composition by known techniques such as freeze-drying or spray-drying for example. Alternatively, bacterial preparations can be bought from specialist suppliers such as DSM, Dupont Danisco, Morinaga, Institut Rosell, Christian Hansen and Valio, already prepared in a suitable form for addition to a composition in powder form.

The composition of the invention may also contain minerals and other micronutrients, understood to be essential in the daily diet and in nutritionally significant amounts. Minimum requirements have been established for certain minerals. Examples of minerals and other nutrients optionally present in the composition of the invention include folic acid, inositol, niacin, biotin, pantothenic acid, choline, calcium, phosphorus, iodine, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended target group.

### Process of Making a Composition

According to a further embodiment, the object underlying the present invention is therefore preferably also solved by a process for preparing a composition as defined herein. In this regard, said process may contain or comprise any of the amounts and ingredients as defined for the inventive composition.

According to a particularly preferred embodiment, the invention relates to a process for the preparation of a composition as described herein, which comprises the step of mixing or dry blending the ingredients as defined herein above to obtain a composition.

Said process, preferably further comprises the steps of
a) carrying out at least one heat treatment step of said mixture obtained after the mixing or dry blending the ingredients; and
b) optionally homogenizing the mixture before or after the heat treatment step.

Advantageously, said process includes steps such as heat treatment and homogenization which result in improved safety and quality of the product. In the compositions of the present invention, compounds sensitive to oxidation, as described above, are advantageously stabilized in such a way that oxidation is prevented or reduced even when the relatively aggressive process steps of heat treatment and homogenization are carried out. Therefore, the composition of the present invention retains good sensory and nutritional properties, as a consequence of limited oxidation of LC-PUFA during heat treatment and homogenization.

The inventive process preferably results in a solid, liquid or semi-liquid/semi-solid composition. When the inventive composition is in solid form, such as a powder, the process should preferably include a drying step, such as a spray-drying, freeze drying or fluid bed agglomeration step. In a preferred embodiment, the composition is in the form of a powder.

### Use of Ferrous Sulphate Monohydrate for the Fortification of a Composition

Ferrous sulphate monohydrate can advantageously be used for the fortification of a composition comprising LC-PUFAs. Such iron source advantageously provides bioavailable iron, while causing little oxidation of the LC-PUFAs.

Ferrous sulphate monohydrate having a bioavailability similar to that of ferrous sulphate heptahydrate, as described above, they are particularly useful to fortify food products.

In another embodiment, the present invention relates to a method for fortifying a composition comprising LC-PUFAs, said method comprising addition to the composition of ferrous sulphate monohydrate.

The composition is as defined in any embodiment of the “composition” section.

In a preferred embodiment, at least 50 wt %, more preferably at least 60 wt %, more preferably at least 70 wt %, more preferably at least 80 wt %, even more preferably at least 90 wt % of the added iron in the composition is in the form of ferrous sulphate monohydrate. Even more preferably, the ferrous sulphate monohydrate is substantially the only added iron source in the composition. Most preferably, the ferrous sulphate monohydrate is the only added iron source in the composition.

A Composition for Use in a Method to Prevent, Reduce and/or Treat Iron Deficiency

The composition of the invention being fortified with ferrous sulphate monohydrate, which is highly bioavailable, as described above, the present invention also relates to a composition for use in a method to prevent, reduce and/or treat iron deficiency in an individual.

### Method for Providing a Nutrition

A method for providing a nutrition to an individual comprising feeding the individual with an edible composition of the invention is also contemplated. The composition used in this method is a food or beverage composition. Preferably, it is a nutritional composition as defined above. Such edible compositions are particularly advantageous for providing a nutrition because they comprise a bioavailable source of iron and only low levels of oxidized LC-PUFAs, which would have reduced nutritional value and undesirable sensory properties.

In an embodiment wherein the composition is in powder form, the method comprises the steps of
a) reconstituting an edible composition in powder form according to any of the embodiments of the invention; and
b) feeding an individual with the reconstituted composition.

In an embodiment, the individual is an individual having an iron deprivation or an individual at risk of developing an iron deprivation. In another embodiment, the individual is an infant, a young child, a woman during pregnancy, during lactation or pre-pregnancy, or an elderly person. More preferably, the individual is an infant, a young child or a woman during pregnancy, during lactation or pre-pregnancy. Most preferably, the individual is a woman during pregnancy, during lactation or pre-pregnancy.

### Method for Preventing or Reducing the Oxidation of Compounds Sensitive to Oxidation

The invention relates to a method for reducing and/or preventing the oxidation of LC-PUFAs in a composition comprising an added iron source, characterized in that ferrous sulphate monohydrate is used as the added iron source. Preferably, the method is further characterized by the fact that ferrous sulphate monohydrate reduces and/or prevents the oxidation of the LC-PUFAs.

In other words, the invention relates to a method for reducing and/or preventing the oxidation of LC-PUFAs in a composition comprising an added iron source, wherein ferrous sulphate monohydrate is used as the added iron source and reduces and/or prevents the oxidation of the LC-PUFAs. Preferably, ferrous sulphate monohydrate reduces the oxidation of LC-PUFAs compared to the oxidation that would be observed with ferrous sulphate heptahydrate being used as the added iron source.

Worded differently, the invention relates to the use of ferrous sulphate monohydrate for reducing and/or preventing the oxidation of LC-PU FA in a composition, preferably in a composition comprising LC-PUFAs and an added iron source. The invention also relates to the use of an iron source consisting of ferrous sulphate monohydrate for reducing and/or preventing the oxidation of LC-PU FA in a composition, preferably in a composition comprising LC-PUFA and an added iron source.

The LC-PUFAs, the added iron source and the composition are as described in any embodiment of the “composition” section.

In a preferred embodiment, the ferrous sulphate monohydrate represents at least 50 wt %, more preferably at least 60 wt %, more preferably at least 70 wt %, more preferably at least 80 wt %, even more preferably at least 90 wt % of the added iron in the composition. More preferably the ferrous sulphate monohydrate is substantially the only added iron source used in the composition. Most preferably, the ferrous sulphate monohydrate is the only added iron source in the composition. In other words, the composition comprises no ferrous or ferric compound added as an iron source in the composition other than ferrous sulphate monohydrate.

The present inventors have shown that by using ferrous sulphate monohydrate instead of other commonly added iron sources, such as ferrous sulphate heptahydrate or dissolved ferrous sulphate in spray-dried form, the oxidation of LC-PUFAs could be prevented or at least significantly reduced.

The added iron source has an important impact on the oxidation of sensitive compounds, whereas the impact of iron sources present as part of an ingredient that is not intended mainly for the purpose of iron supplementation is smaller, as the latter iron sources are often less reactive and iron provided by the latter iron source is usually provided in much lower amounts than that provided by the added iron source.

The level of oxidation of LC-PUFAs can be assessed using well-known techniques, including the analysis of markers of oxidation. It can also be assessed using sensory experiments. Preventing or reducing oxidation of LC-PUFAs is evaluated by reduction of off-taste, such as rancidity, fishiness, metallic, painty, fried fat, etc. in the composition, when compared to a composition comprising the same ingredients but another kind of iron source. Such an off-flavour can be tested and verified by a skilled person following accepted standards of sensory testing, such as the preference test.

### Further Second Medical Uses

The composition of the present invention can be used for prevention, amelioration or treatment of a disease or disorder as defined herein. As used herein, the term “a disorder” or “a disease” refers to any derangement or abnormality of function; a morbid physical or mental state. See Dorland's Illustrated Medical Dictionary, (W.B. Saunders Co. 27th ed. 1988). Such diseases or disorders may be selected from malnutrition, metabolic diseases, neurodegenerative diseases, Alzheimer disease/cognitive impairment, Parkinson's disease, neurological diseases, Amyotrophic lateral sclerosis, Traumatic brain injury, Hypoxic/ischemic brain injury, Autism, ADHD (Attention Deficit Hyperactivity Disorder), Depression, Headaches, Migraine Headaches, Narcolepsy, GLUT-1 deficiency, Pyruvate Dehydrogenase (PDH) deficiency, phosphofructokinase (PFK) deficiency, Glycogenosis type V (McArdle disease), Cardiac ischemia, Rett syndrome, Tuberous Sclerosis, Diabetes and Cancer (astrocytornas, prostate, gastric, renal, head and neck), preferably for use in the prevention, amelioration or treatment of malnutrition, metabolic diseases, neurodegenerative diseases, preferably as a nutritional supplement. The composition is preferably used as a nutritional composition or supplement.

The composition of the present invention can also be used for the promotion of the development of the nervous system and/or of the retina, and/or in the promotion and/or improvement of the mental performance, behavioural and visual functions of an infant or a child.

For the purpose of the present invention, mental performance is for example intended as cognitive and intellectual performance, memory, as well as language ability of an infant or child. Development of the nervous system is intended to include for example brain and neuronal development.

The composition of the present invention can further be used to strengthen immunity, including the development of gut microflora.

The composition of the present invention can be used for reducing the risk of the development of overweight, obesity and insulin resistance.

The advantageous effects of the inventive composition as described above are preferably accomplished by administering an effective amount of a composition according to the present invention to a subject in need thereof. Preferably, such a composition is to be administered once daily, preferably twice daily, more preferably three times daily, wherein during administration preferably at least one unit or dose for administration is provided, as defined herein. Upon administration, preferably the total amount of energy to be administered per day is as defined before. As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human, more preferably selected from an infant, a child or an adult. The term “effective amount” of a composition of the present invention refers to an amount of the compound of the present invention that will elicit the biological or medical response of a subject, enhance development of organs or functions of a subject, or ameliorate symptoms, slow or delay disease progression, or prevent a disease, etc. Preferably, such an “effective amount” is a packaged dose or unit as obtained as described herein.

The present invention will now be described in further details by the way of the following examples.

### Example 1: Effect of the Iron Source on the Fishy Off Flavour of Skimmed Milk Fortified with DHA and Diverse Iron Sources

The intensity of fishy off-flavour was assessed in a model composition comprising DHA and different sources of iron. Test samples were prepared with skimmed milk, DHA and diverse iron sources (Samples 1 to 5). Such sample had the composition provided in Table 1. In each sample the iron source was added in an amount such as to provide 13 mg of Fe²⁺ per 100 g of sample and the DHA powder was added in an amount such as to provide 500 mg of DHA per 100 g of sample.

TABLE 1
Composition of Samples 1 to 5

| | Amount in Sample # [g] | | | | |
|---|---|---|---|---|---|
| Ingredient | #1 | #2 | #3 | #4 | #5 |
| Ferrous sulphate spray- | 1.31 | 0.00 | 0.00 | 0.00 | 0.00 |
| dried¹⁾ | | | | | |
| Ferrous sulphate | 0.00 | 1.63 | 0.00 | 0.00 | 0.00 |
| monohydrate premix - | | | | | |
| micronized powder²⁾ | | | | | |
| Ferrous sulphate | 0.00 | 0.00 | 1.63 | 0.00 | 0.00 |
| monohydrate premix - | | | | | |
| fine powder³⁾ | | | | | |
| Ferrous sulphate | 0.00 | 0.00 | 0.00 | 1.63 | 0.00 |
| monohydrate premix⁴⁾ | | | | | |
| Ferrous sulphate | 0.00 | 0.00 | 0.00 | 0.00 | 1.63 |
| monohydrate premix- | | | | | |
| coarse powder⁵⁾ | | | | | |
| Skimmed milk powder | 230.83 | 230.51 | 230.51 | 230.51 | 230.51 |
| DHA in powder form⁶⁾ | 17.86 | 17.86 | 17.86 | 17.86 | 17.86 |
| Total | 250.00 | 250.00 | 250.00 | 250.00 | 250.00 |

¹⁾TE218; origin Nestlé; material number 40800220; obtained by dissolving ferrous sulphate in water at pH 2 and spray-drying in a maltodextrin matrix. This iron source contains 8.4 wt % of Fe²⁺
²⁾Ferrous sulphate monohydrate dry-mixed with maltodextrin in the form of a micronized powder; USP 36; origin Nestlé; material number 103508539
³⁾Ferrous sulphate monohydrate dry-mixed with maltodextrin in the form of a fine powder; Origin: Nestlé; matrial number: 103508621
⁴⁾Ferrous sulphate monohydrate dry-mixed with maltodextrin in the form of a coarse powder; origin: Nestlé; material number: 103327677
⁵⁾Origin: Nestlé; material number: 103508624
⁶⁾NIF powder; origin: Friesland Campina Kievit.

Each of the samples 1 to 5 was prepared as follows.
1. The skimmed milk was weighed in a 1 L container
2. The ferrous sulphate premix was weighed in a 250 mL container
3. An amount of 10 g of skimmed milk was added to the ferrous sulphate premix in the 250 mL container.
4. The 250 mL container was closed and the content was mixed for about 5 s.
5. The OHA was weight in a separate 250 mL container.
6. An amount of 10 g of the skimmed milk powder was added to the DHA in the 250 mL container.
7. The container with the OHA was closed and the content was mixed for about 5 s.
8. The contents of both 250 mL containers was then poured into a clean 1 L container
9. An amount of 10 g of skimmed milk of step 1 was then poured into each of the 250 mL containers to dry clean the container and the skimmed milk was then added to the 1 L container of step 8 containing the ferrous sulphate premix and the DHA.
10. The rest of the skimmed milk of step 1) was then added to the mixture of step 9.
11. The 1 L container of step 10 was then closed and placed in a tumbler mixer for 10 minutes.

Each sample was then packed separately in aluminium bags without gassing.

A Reference was prepared by packing pure skimmed milk powder in an aluminium bag as described above.

All samples were stored for 2.5 weeks at 30° C.

After storage, the samples were subjected to a sensory evaluation by a panel of trained panelists. Samples to be tasted by panelists were prepared by dissolving 70 g of each Sample (Samples 1 to 5 and Reference Sample) in 500 mL of Vittel water at 40° C. Samples 1 to 5 and the Reference sample were presented at once to the panelists and compared to the Reference Sample. The panelists were requested to rate the fishiness of each of the test Samples and the Reference over the Reference according to the following scale:

0: same as REF
+1: just a little more intense than REF
+2: slightly more than REF
+3: clearly more intense than REF
+4: much more intense than REF
+5: very much more intense than REF.

This scale was selected as it was not expected to have samples with less off-flavour than the reference comprising no DHA.

The statistical analysis of the results was performed using the Duncan test (α=0.05). The results of the sensory evaluation are provided in Table 2 below. Samples connected by the same black line are not significantly different (p<0.5).

TABLE 2
results of the evaluation of fishy off-flavour intensity
in Samples 1 to 5 and in the Reference sample.

| | Mean fishiness | | | | | |
|---|---|---|---|---|---|---|
| Sample | score | Statistical significance | | | | |
| Reference | 0.04 | | X | | | |
| Sample 5 (invention) | 0.41 | | X | X | | |
| Sample 3 (invention) | 0.62 | | | X | X | |
| Sample 4 (invention) | 0.76 | | | X | X | |
| Sample 2 (invention) | 0.97 | | | | X | |
| Sample 1 (comparative) | 1.48 | | | | | X |

The fishiness of Sample 5 was not statistically significant from the reference with no DHA at all. All other test samples are just a little fishy but Samples 3 and 4 were not significantly different from Sample 5. Also, sample 2 was not significantly different from Samples 3 and 4. Sample 1 (according to the prior art) was in contrast significantly more fishy than all other Samples. These results demonstrate a lower fishy off-flavour when ferrous sulphate monohydrate is used compared to when ferrous sulphate spray-dried in an amorphous matrix is used. The results also show that this result is achieved with diverse grades of ferrous sulphate monohydrate, all in crystalline form, and namely with different crystal sizes.

At the time of opening the packaging of each Sample, the residual amount of oxygen in the aluminium bags was analysed, in order to assess if a difference of fishiness is due to a reduced oxidation of the DHA. The results are provided in Table 3 below.

TABLE 3
Residual amount of oxygen in the aluminium
bags of Samples 1 to 5 and of the Reference

| Sample | Residual O₂ [%] |
|---|---|
| Sample 1 (comparative) | 17.8 |
| Sample 2 (invention) | 20.3 |
| Sample 3 (invention) | 20.5 |
| Sample 4 (invention) | 20.5 |
| Sample 5 (invention) | 20.6 |

Residual oxygen indicating an oxidation process in the respective samples correlates with the intensity of the fishy off-flavour: the less oxygen, the more fishy the sample is.

1. A composition comprising LC-PUFAs and an iron source, the iron source is ferrous sulphate monohydrate.

2. A composition according to claim 1, the composition is a nutritional composition.

3. A composition according to claim 1, the iron source is present in an amount such as to provide 6 to 50 mg of iron per 100 g of composition.

4. A composition according to claim 1, the composition comprises 10 to 1000 mg of LC-PUFAs per 100 g, based on the total dry weight of the composition.

5. A composition according to claim 1, the composition comprises 10 to 750 mg of LC-PUFAs per 100 g, based on the total dry weight of the composition.

6. A composition according to claim 1, the composition comprises 10 to 500 mg of LC-PUFAs per 100 g, based on the total dry weight of the composition.

7. (canceled)

8. A method of preventing, reducing and/or treating iron deficiency in an individual in need of same comprising administering a composition comprising LC-PUFAs and an iron source, the iron source is ferrous sulphate monohydrate.

9-12. (canceled)

13. Method for use in the promotion of the development of the nervous system and/or of the retina, in the promotion and/or improvement of the mental performance, behavioural and visual functions of an infant or a child, for strengthening immunity, including the development of gut microflora, and/or for reducing the risk of the development of overweight, obesity and insulin resistance comprising the step of administering to an individual in need of same a composition comprising LC-PUFAs and an iron source, the iron source is ferrous sulphate monohydrate.
